(19) United States
(12) Patent Application Publication
(10) Pub. No.: US 2021/0267879 A1
(43) Pub. Date: Sep. 2, 2021

(54) ANTI-ITCH AND SKIN MOISTURIZING COMPOSITIONS

(71) Applicants: KEENAN BEASLEY, BROOKLYN, NY (US); KENYATA MARTIN, CHICAGO, IL (US)

(72) Inventors: KEENAN BEASLEY, BROOKLYN, NY (US); KENYATA MARTIN, CHICAGO, IL (US)

(21) Appl. No.: 16/805,760

(22) Filed: Feb. 29, 2020

(51) Int. Cl. A61K 8/99; A61K 8/9728; A61K 8/368; A61K 8/9783; A61Q 19/00

(52) U.S. Cl. CPC A61K 8/99; A61K 8/9728; A61K 8/368; A61K 2800/75; A61Q 19/00; A61K 2800/10; A61K 8/9783

(57) ABSTRACT

The invention provides anti-itch compositions applicable to a user's scalp. In particular, the anti-itch compositions are particularly suitable for naturally curly hair types and also for users who use non-native hair (weaves), braided hair substantially comprising non-native hair and wigs. One composition is a fast-acting anti-itch remedy and another is a scalp moisturizer that conditions and enhances the scalp.

### FIELD OF THE INVENTION

The invention provides anti-itch and moisturizing compositions applicable to a user's skin. In particular, the anti-itch and moisturizing compositions are particularly suitable for scalps of naturally curly hair types and also for users who use non-native hair (weaves), braided hair substantially comprising non-native hair and wigs.

### BACKGROUND OF THE INVENTION

Throughout antiquity, mankind has sought alter his or her appearance. This is especially true in the presentation of hair. The ancient Egyptians, provide thousands of years of testament in their art and architecture showing the importance of hair types and styles. In fact, the inhabitants of Ancient Kemet (i.e., KMT or Egypt) were renowned throughout the world for their use of braided hair and wigs. The hair types of these indigenous ancient people is known to have been naturally curly.

In modern times, hair care, its uses and its styling is a multi-billion dollar world wide business. In years past, woman and men of varying ethnicities styled their hair to Western norms and standards of beauty. In more recent times, men and women of non-European ethnic background have delved heavily into natural hairstyles with their own hair, weaved hair (I.e., natural and non-natural), hair for wigs and so forth.

As natural hairstyles have developed and in particular the use of hair weaved into a wearer's own natural hair, problems have developed. For example, the scalps of such wearer's can tend to dry out faster thereby suffering from a suitable lack of moisture. Also, the ability to get moisturizing products to a wearer's scalp especially where weaved hair is used can be difficult. Lastly, the hair care industry, which has mostly focused on European hair and scalp types, has been slow to react to the long felt, unmet needs of caring for the hair and scalps of non-European people.

What is therefore needed is a product or products that enable curly, natural hair wearers, those who wear weaves, and those who wear wigs to moisturize their scalp and/or hair and to prevent a loss of moisture due to either hair style or use of non-native hair to the wearer. The embodiments presented herein serve to solve these and more problems and will be fully elucidated herein.

### SUMMARY OF THE INVENTION

Accordingly, the invention provides a fast-acting anti-itch scalp composition that comprises a pseudoalteromonas ferment extract; sodium salicylate; cordyceps sinensis extract; and trametes versicolor extract.

The anti-itch scalp composition herein may further comprise an extract of mentha piperita. The anti-itch scalp composition may further comprise oil of mentha piperita. As an additional ingredient, the anti-itch scalp composition herein may also comprise perfume.

The anti-itch scalp composition herein provides anti-itch relief upon application within about fifteen minutes. The anti-itch scalp composition herein provides anti-itch relief upon application preferably within about ten minutes. The anti-itch scalp composition herein provides anti-itch relief upon application most preferably within about five minutes.

In practice, the anti-itch scalp composition herein provides anti-itch relief to a scalp for at least about eight continuous hours. More preferably, the anti-itch scalp composition herein provides anti-itch relief to a scalp for at least about sixteen continuous hours. Most preferably, the anti-itch scalp composition herein provides anti-itch relief to a scalp for at least about twenty-four continuous hours.

The anti-itch scalp composition herein is particularly formulated and suitable for curly, natural hair types ranging from 3A to 4C of the hair type guide. Also, the anti-itch scalp composition herein is suitable for head types comprising weaved hair, braided hair using non-native hair, and/or head types upon which wigs are worn.

Another scalp enhancing composition provided herein is a scalp moisturizing spray composition that comprises a pseudoalteromonas ferment extract, Camelia Sinensis leaf extract, and sodium salicylate. Like the anti-itch composition noted hereinabove, the hair moisturizing spray herein provides an anti-itch benefit. The hair moisturizing spray composition may further comprise Argania Spinosa Kernel oil and perfume.

The scalp moisturizing composition herein is particularly formulated and suitable for curly, natural hair types ranging from 3A to 4C of the hair type guide. Also, the anti-itch scalp composition herein is suitable for head types comprising weaved hair, braided hair using non-native hair, and/or head types upon which wigs are worn.

The scalp moisturizing composition herein also provides anti-itch relief upon application to a user's scalp within about fifteen minutes. The anti-itch scalp composition herein provides anti-itch relief upon application preferably within about ten minutes. The anti-itch scalp composition herein provides anti-itch relief upon application most preferably within about five minutes.

An anti-itch scalp moisturizing complex, comprising a Pseudoalteromonas ferment extract and a plant extract suitable for use in moisturizing human skin. The complex is particularly applicable for very dry skin and particularly for the scalps of hair types ranging from 3A to 4C, wig wearers, and weave wearers.

### DETAILED DESCRIPTION OF THE INVENTION

One of the inventions herein provides a fast-acting anti-itch scalp composition that comprises a pseudoalteromonas ferment extract; sodium salicylate; cordyceps sinensis extract; and trametes versicolor extract.

The anti-itch scalp composition herein may further comprise a leaf extract of mentha piperita (i.e., Peppermint). The anti-itch scalp composition may further comprise an oil of mentha piperita (i.e., Peppermint). As an additional ingredient, the anti-itch scalp composition herein may also comprise perfume.

The anti-itch scalp composition herein provides anti-itch relief upon application within about fifteen minutes. The anti-itch scalp composition herein provides anti-itch relief upon application preferably within about ten minutes. The anti-itch scalp composition herein provides anti-itch relief upon application most preferably within about five minutes.

It has been discovered that the combination of pseudoalteromonas ferment extract; sodium salicylate; cordyceps sinensis extract; and trametes versicolor extract operate together to relieve particular scalp types to relieve scalp itching for a desired amount of time and to start working quickly upon application thereof.

This embodiment is particularly effective for scalp types of curly hair. It is also particularly effective for scalp types of curly hair and users who weave non-native hair to their native hair and/or attach non-native hair to their scalps. By the term “non-native hair” it is meant herein hair, whether synthetic or human, that a user or wearer did not herself or himself grow. By the term “native hair” it is meant herein hair that a user or wearer has himself or herself grown.

The fast-acting anti-itch scalp composition herein is also highly efficacious against scalp itch for users that wear wigs. Wigs, especially when worn daily or consistently, can tend to cause dry scalp conditions that lead to scalp itch. Use of the anti-itch scalp composition herein before, during and/or after a wig is worn can protect against scalp itch, i.e., if applied either before or during wig wear, and/or enable recovery from scalp itch when the composition is applied after a wig is worn and later removed.

The Pseudoalteromonas ferment extract used herein is a biotechnologically derived ingredient that is produced by the fermentation of a marine bacteria living in the cold Antarctic Ocean. It is generally known to encompass at least two properties. First, it provides skin-protecting and anti-aging abilities. Second, it functions as an anti-itch agent that can help to protect skin from dryness and redness whether due to cold weather or some other condition. Pseudoalteromonas ferment extract is also known to promote skin regeneration and smoother skin surface by stimulating protein synthesis in the skin. More specifically, these proteins are type I and IV collagen and elastin which are critical in the maintenance of healthy skin.

Structurally, Pseudoalteromonas ferment extract is a peptide that is classified as a protein derivative, and contains the following three amino acid residues: glycine, histidine and lysine. It is a strain of bacteria isolated from mud found on King George Island in Antarctica.

Sodium salicylate is a sodium salt of salicylic acid. It can be prepared from sodium phenolate and carbon dioxide under higher temperature and pressure. Historically, it has been synthesized by refluxing methyl salicylate with an excess of sodium hydroxide. It's chemical formulation is C₇H₅NaO₃. Typically, sodium salicylate is used to treat minor aches and pains of the muscles/joints (e.g., arthritis, backache, sprains). Methyl salicylate is known as a counterirritant. It works by causing the skin to feel cool and then warm. It has been discovered to provide important relief to scalp itch especially when combined with the Pseudoalteromonas ferment extract.

Cordyceps sinensis extract, another ingredient of the fast-acting anti-itch scalp composition herein, provides a central benefit of fighting inflammation, for example, the kind of inflammation common to itchy, irritated skin. There are many bioactive components of Cordyceps sinensis extracted therefrom including nucleoside, polysaccharide, sterol, protein, amino acid, and polypeptide.

Trametes versicolor, also known as Coriolus versicolor and Polyporus versicolor, is a common polypore mushroom found throughout the world. Meaning ‘of several colors’, versicolor reliably describes this fungus that displays different colors. Its colloquial name is the ‘turkey tail mushroom’.

Trametes versicolor is well known to contain an abundance of antioxidants, polysaccharopeptides (immune-boosting), immune function improvement for some with cancer, enhancement of gut health, and the reduction of inflammation. Particularly for its anti-inflammation benefits, it highly useful by itself but especially in combination with the other three main ingredients named herein; i.e., Pseudoalteromonas ferment extract, sodium salicylate and Cordyceps sinensis extract.

Each of the four main ingredients in the fast-acting anti-itch scalp composition herein each possess anti-itching properties either due to their native anti-inflammatory characteristics and/or because it specifically functions to dull itching in skin, whether inflamed or not. All four of these ingredients work together to produce both fast acting relief and a long lasting itch remedy for specific hair types, i.e., those ranging from 3A to 4C, and the scalps thereof.

In practice, the anti-itch scalp composition herein provides anti-itch relief to a scalp for at least about eight continuous hours. More preferably, the anti-itch scalp composition herein provides anti-itch relief to a scalp for at least about sixteen continuous hours. Most preferably, the anti-itch scalp composition herein provides anti-itch relief to a scalp for at least about twenty-four continuous hours.

The anti-itch scalp composition herein can be provided in multiple concentrations. The concentration level can tend to impact a) how quickly it acts to halt scalp itch b) how long scalp itch is ceased. Concentration levels may range from mildly concentrated to mediumly concentrated to highly concentrated. Choice of concentration is manufacturer's choice and itself forms no part of the invention.

The anti-itch scalp composition herein is particularly formulated and suitable for curly, natural hair types ranging from 3A to 4C of the hair type guide. Also, the anti-itch scalp composition herein is suitable for head types comprising weaved hair, braided hair using non-native hair, and/or head types upon which wigs are worn as noted hereinabove.

Importantly, the addition of both mentha piperita (peppermint) leaf extract and mentha piperita oil to the anti-itch composition adds further synergistic effects to the composition. First, both the peppermint extract and oil add a cooling sensation to inflamed and irritated nerve endings thereby enabling a fast-acting cessation of the most immediate surface itching of a scalp. Second, the peppermint extract and oil serve to promote hair health and protect against dandruff or flakes which may be resident from long term scalp itch as, in the instance, a weave is worn for two or more weeks upon a wearer's head.

Another scalp enhancing composition provided herein is a scalp moisturizing spray composition that comprises a pseudoalteromonas ferment extract, Camelia Sinensis leaf extract, and sodium salicylate. Like the anti-itch composition noted hereinabove, the hair moisturizing spray herein provides an anti-itch benefit. The hair moisturizing spray composition may further comprise Argania Spinosa Kernel oil and perfume.

The chief objective of the scalp enhancing composition is to keep it moisturized and to condition it to fight against scalp itch for those scalp types of curly hair, weaved hair and those that regularly wear wigs. For the subject user type, a user would be directed to daily apply the scalp enhancing composition directly to his or her scalp as part of the user's regular hair care regiment.

Within the anti-itch scalp composition herein the Pseudoalteromonas ferment extract exists within the composition in a range from about 0.5 wt. percent to about 10 wt. percent. Within the anti-itch scalp composition herein the sodium salicylate exists within the composition in a range from about 0.5 wt. percent to about 10 wt. percent. Within the anti-itch scalp composition herein the cordyceps sinensis extract exists within the composition in a range from about 0.5 wt. percent to about 10 wt. percent. Within the anti-itch scalp composition herein the Trametes versicolor extract exists within the composition in a range from about 0.5 wt. percent to about 10 wt. percent.

The scalp moisturizing composition herein is particularly formulated and suitable for curly, natural hair types ranging from 3A to 4C of the hair type guide. Also, the anti-itch scalp composition herein is suitable for head types comprising weaved hair, braided hair using non-native hair, and/or head types upon which wigs are worn.

The scalp moisturizing composition herein also provides anti-itch relief upon application to a user's scalp within about fifteen minutes. The anti-itch scalp composition herein provides anti-itch relief upon application preferably within about ten minutes. The anti-itch scalp composition herein provides anti-itch relief upon application most preferably within about five minutes.

The ingredients of the scalp moisturizing composition herein operate to produce the synergistic effects of scalp conditioning, scalp enhancement, anti-itch scalp relief and anti-itch scalp prevention. By the term “scalp conditioning” it is meant herein the moisturizing composition's ability to moisturize and thereby condition the skin of the scalp. By the term “scalp enhancement” it is meant herein the moisturizing composition's ability to increase and secure the overall health of one's scalp, i.e., the skin thereof.

Camelia Sinensis leaf extract is commonly known as green tea extract. Its chemical formulation is C₅₀H₅₀N₄O₂₆. Camellia sinensis (or tea plant) is used to make most traditional caffeinated teas, including black tea, white tea, oolong tea, and green tea. This plant originated near the southwest region of China as an evergreen forest shrub. Camellia sinensis has a long list of potential health benefits including the ability to regulate blood sugar and blood pressure, boost the immune system, prevent ulcers, control inflammation, viral colds and flu, prevents gum disease, cavities, and bad breath.

It is the Camelia Sinensis leaf extract's ability to control or reduce inflammation that makes it such a useful ingredient within the scalp moisturizing composition herein. Its use in combination with pseudoalteromonas ferment extract and sodium salicylate maximizes its efficacy for its anti-inflammation benefits and thereby its anti-itch benefit when applied to a user's scalp.

The addition of Argania spinosa kernel oil adds even more benefit to the moisturizing composition. Argania spinosa kernel oil is an oil rich in essential fatty acids, vitamin E and other vital nutrients. In hair care products, it helps to smooth frizz and split ends as well as provide softness. In skin care products, it nourishes, protects elasticity and reduces the appearance of fine lines and wrinkles.

The Argania spinosa kernel oil herein acts as a skin care product since it is meant to be applied directly to a user's scalp. By such application, the moisturizing application thereby provides all of the aforementioned benefits plus further protects the elasticity of the scalp and reduces the appearance of fine lines and wrinkles. Typically, the oil from argantree kernels contains about 45-55% oleic acid, 28-36% linoleic acid, 10-15% palmitic acid and 5-7% stearic acid.

Argan oil has a relative density at 20° C. (68° F.) ranging from 0.906 to 0.919. Argan oil contains tocopherols (vitamin E), phenols, carotenes, squalene, and fatty acids, (80% unsaturated fatty acids) The main natural phenols in argan oil are caffeic acid, oleuropein, vanillic acid, tyrosol, catechol, resorcinol, (−)-epicatechin and (+)-catechin.

The ideal application for the scalp moisturizing composition is in spray form through a spray bottle. Like the anti-itch scalp composition, the scalp moisturizing composition can be provided in multiple concentrations. The concentration level can tend to impact a) how quickly it acts to halt scalp itch b) how long scalp itch is ceased. Concentration levels may range from mildly concentrated to mediumly concentrated to highly concentrated. Choice of concentration is manufacturer's choice and itself forms no part of the invention.

Though application by spray is ideal, it is not the only way to apply the scalp moisturizing composition herein. It can also be applied by one more types of applicators, in a lotion, and by an ionizer.

Within the scalp moisturizing composition herein the Pseudoalteromonas ferment extract exists within the composition in a range from about 0.5 wt. percent to about 10 wt. percent. Within the scalp moisturizing composition herein the camelia sinensis leaf extract exists within the composition in a range from about 0.5 wt. percent to about 10 wt. percent. Within the scalp moisturizing composition herein the sodium salicylate exists within the composition in a range from about 0.5 wt. percent to about 10 wt. percent.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A fast-acting anti-itch scalp composition, comprising: (SOOTHE ME™)
a. Pseudoalteromonas ferment extract;
b. Sodium salicylate;
c. Cordyceps sinensis extract; and
d. Trametes versicolor extract.

2. The anti-itch scalp composition of claim 1 further comprising extract of mentha piperita.

3. The anti-itch scalp composition of Claim 1 further comprising oil of mentha piperita

4. The anti-itch scalp composition of claim 1 further comprising perfume.

5. The anti-itch scalp composition of Claim 1 providing anti-itch relief within about fifteen minutes.

6. The anti-itch scalp composition of claim 1 providing anti-itch relief for at least about eight continuous hours.

7. The anti-itch scalp composition of claim 1 providing anti-itch relief for at least about sixteen continuous hours.

8. The anti-itch scalp composition of claim 1 providing anti-itch relief for at least about twenty-four continuous hours.

9. The anti-itch scalp composition of Claim 1 wherein said composition is suitable for natural hair types ranging from 3A to 4C.

10. The anti-itch scalp composition of claim 1 wherein said composition is suitable for head types with weaved hair.

11. The anti-itch scalp composition of claim 1 wherein said composition is suitable for head types with braided hair using non-native hair.

12. The anti-itch scalp composition of claim 1 wherein said composition is suitable for head types upon which wigs are worn.

13. The scalp moisturizing spray composition, comprising:
a. Pseudoalteromonas ferment extract;
b. Camelia Sinensis leaf extract; and
c. Sodium salicylate.

14. The scalp moisturizing spray composition of claim 13 further comprising Argania Spinosa Kernel oil.

15. The scalp moisturizing spray composition of claim 13 further comprising perfume.

16. The scalp moisturizing spray composition of claim 13 wherein said composition is suitable for natural hair types ranging from 3A to 4C.

17. The scalp moisturizing spray composition of claim 13 wherein said composition is suitable for head types with weaved hair.

18. The scalp moisturizing spray composition of claim 13 wherein said composition is suitable for head types with braided hair using non-native hair,

19. The scalp moisturizing spray composition of claim 13 wherein said composition is suitable for head types upon which wigs are worn.

20. An anti-itch scalp moisturizing complex, comprising:
a. Pseudoalteromonas ferment extract;
b. Sodium salicylate; and
c. Plant extract suitable for use in moisturizing human skin,
Said complex being applicable to hair types ranging from 3A to 4C, wig wearers, and weave wearers.
